(19) United States
(12) Patent Application Publication
(10) Pub. No.: US 2023/0314579 A1
(43) Pub. Date: Oct. 5, 2023

(54) ULTRASONIC DIAGNOSTIC APPARATUS

(71) Applicant: FUJIFILM Healthcare Corporation, Kashiwa-shi (JP)

(72) Inventor: Hiroshi Kuribara, Chiba (JP)

(21) Appl. No.: 18/117,359

(22) Filed: Mar. 3, 2023

(30) Foreign Application Priority Data
Mar. 29, 2022 (JP) 2022-053837

(51) Int. Cl. G01S 7/52; A61B 8/00

(52) U.S. Cl. CPC G01S 7/52033; A61B 8/4488; A61B 8/54

(57) ABSTRACT

A plurality of amplifiers amplify a plurality of received signals in accordance with a gain function that varies with a depth. A maximum amplitude detector detects a maximum amplitude at each depth from among the plurality of received signals. A gain function corrector corrects the gain function based on the maximum amplitude at each depth. A corrected gain function is applied to the plurality of amplifiers. A compensator performs gain compensation with respect to beam data.

### CROSS REFERENCE TO RELATED APPLICATION

This application claims priority to Japanese Patent Application No. 2022-053837 filed on Mar. 29, 2022, which is incorporated herein by reference in its entirety including the specification, claims, drawings, and abstract.

### TECHNICAL FIELD

The present disclosure relates to an ultrasonic diagnostic apparatus, and more particularly to processing of a plurality of received signals.

### BACKGROUND

Prior to ultrasonic inspection of a subject using an ultrasonic diagnostic apparatus, a gain function is selected in accordance with an ultrasonic probe and transmission frequencies, for example. The gain function is a function that dynamically changes a gain, a ratio of an output value to an input value, in accordance with the depth. The gain function that is selected is applied to a plurality of analog amplifiers. The analog amplifiers amplify a plurality of received signals according to the applied gain function. During the ultrasonic inspection, the selected gain function is used continuously.

JP H07-236637 A (Document 1) discloses an ultrasonic diagnostic apparatus including an automatic gain controller that adjusts an analog gain based on an ultrasound image. Document 1, however, does not disclose adjustment of the analog gain based on a plurality of received signals before phase alignment and summing.

### SUMMARY

Amplifying a received signal in accordance with the gain function eliminates or reduces dependency of the received signal intensity on the depth. However, the presence of a strong reflector within a living body would cause an excessively large amplitude of the amplified received signal, yielding saturation of the received signal. Meanwhile, ultrasonic attenuation within a living body that is larger than expected would cause an excessively small amplitude of the amplified received signal, lowering the sensitivity.

An aspect of the disclosure is therefore aimed toward amplifying received signals without causing an excessively large or small amplitude. A further aspect of the disclosure is aimed toward matching the gain function to situations during operation of an ultrasonic diagnostic apparatus.

In accordance with an aspect of the disclosure, an ultrasonic diagnostic apparatus includes a plurality of amplifiers configured to amplify a plurality of received signals in accordance with a gain function that varies with a depth and to output a plurality of amplified received signals, a beamformer configured to apply delay to the plurality of amplified received signals and then add up a plurality of delayed received signals, thereby generating beam data, a detector configured to detect a representative value sequence comprising a plurality of representative values arranged in a depth direction, based on the plurality of amplified received signals or the plurality of delayed received signals, and a corrector configured to correct an original gain function based on the representative value sequence to thereby generate a corrected gain function. The corrected gain function is applied to the plurality of amplifiers as the gain function.

### BRIEF DESCRIPTION OF DRAWINGS

Embodiments of the present disclosure will be described based on the following figures, wherein:

FIG. 1 is a block diagram illustrating an example configuration of an ultrasonic diagnostic apparatus according to an embodiment;

FIG. 2 illustrates a plurality of received signals output from a plurality of transducers;

FIG. 3 illustrates amplification of a received signal based on a gain function;

FIG. 4 illustrates detection of a maximum amplitude sequence;

FIG. 5 illustrates generation of a correction gain function and a compensation function;

FIG. 6 illustrates two transmission and reception operations adjacent in time;

FIG. 7 illustrates two transmission and reception operations having a spatial correspondence;

FIG. 8 is a block diagram illustrating a first modification example;

FIG. 9 is a block diagram illustrating a second modification example; and

FIG. 10 is a block diagram illustrating a third modification example.

### DESCRIPTION OF EMBODIMENTS

Embodiments of the disclosure will be described by reference to the drawings.

### (1) Summary of Embodiments

An ultrasonic diagnostic apparatus according to an embodiment includes a plurality of amplifiers, a beamformer, a detector, and a corrector. The amplifiers amplify a plurality of received signals in accordance with a gain function that varies with a depth and output a plurality of amplified received signals. The beamformer applies delay to the plurality of amplified received signals and then adds up a plurality of delayed received signals, thereby generating beam data. The detector detects a representative value sequence composed of a plurality of representative values arranged in a depth direction, based on the plurality of amplified received signals or the plurality of delayed received signals. The corrector corrects an original gain function based on the representative value sequence to thereby generate a corrected gain function. The corrected gain function is applied to the plurality of amplifiers as the gain function.

The above configuration references a plurality of received signals that have been amplified by a plurality of amplifiers, and adaptively corrects a gain function based on the reference result. This enables the gain function to adapt to situations within a living body. It is therefore possible to prevent or reduce saturation of the received signal or lowering of the sensitivity. As the above configuration partially corrects the existing gain function rather than generating a new gain function, the existing configuration can be used and the possibility of excessive correction can be reduced.

According to the embodiment, each of the plurality of representative values is a maximum amplitude detected at each detecting timing, and the representative value sequence is a maximum amplitude sequence. In addition to the maximum amplitude, the representative value may also be a mean value (average amplitude) or a minimum value (minimum amplitude), for example. Referencing the maximum value or the maximum amplitude as the representative value effectively reduces saturation of each received signal. The gain function corrector, which will be described below, is an example of the corrector. The detector samples a plurality of amplitudes from a plurality of received signals at each detection timing or each depth, and compares the plurality of amplitudes with each other, thereby specifying the maximum amplitude. The plurality of received signals which have not been delayed yet may be sampled or the delayed received signals may be sampled. To prevent creation of a vibrating state at the time of correcting the gain function, the correction amount may be regulated or the feedback velocity or responsivity may be controlled.

According to the embodiment, the corrector determines at least an excessively large gain based on the maximum amplitude sequence, and, in response to determination of the excessively large gain, applies downward correction to the original gain function. This configuration reduces the possibility of saturation of a received signal in any reception channel. The corrector further determines an excessively small gain based on the maximum amplitude sequence, and, in response to determination of the excessively small gain, applies upward correction to the original gain function. This configuration prevents lowering of the quality of the beam data.

In the embodiment, the corrector determines the excessively large gain based on comparison of respective maximum amplitudes forming the maximum amplitude sequence with a first threshold value; and determines the excessively small gain based on comparison of respective maximum amplitudes comprising the maximum amplitude sequence with a second threshold value that is smaller than the first threshold value. The first threshold value and the second threshold value may be varied automatically or manually in accordance with various conditions. The first threshold value and the second threshold value may be changed in accordance with the depth.

In the embodiment, the corrected gain function generated based on a plurality of received signals obtained in the previous transmission and reception is applied to a plurality of received signals obtained in the current transmission and reception. This configuration enables adaptive correction of the gain function while performing normal transmission and reception.

In the embodiment, the previous transmission and reception and the current transmission and reception are continuous in time. Typically, two transmission and reception operations adjacent in time are also adjacent in space. The received signal sequence obtained in the previous transmission and reception and the received signal sequence obtained in the current transmission and reception can therefore be treated as being identical. The above configuration generates and applies the corrected gain function based on such a precondition.

The ultrasonic diagnostic apparatus according to the embodiment further includes a generator and a compensator. The generator generates a compensation function based on the representative value sequence, and the compensator applies the gain compensation to the beam data in accordance with the compensation function. This configuration compensates a change in the amplitude of the beam data caused by correction of the gain function.

In the embodiment, the generator generates the compensation function such that the amplitude of the beam data increases in response to application of downward correction to the original gain function and such that an amplitude of the beam data decreases in response to application of upward correction to the original gain function. Provided that a difference between the original gain function and the corrected gain function is represented as a correction function, the correction function, and the compensation function are complementary to each other.

### (2) Details of Embodiments

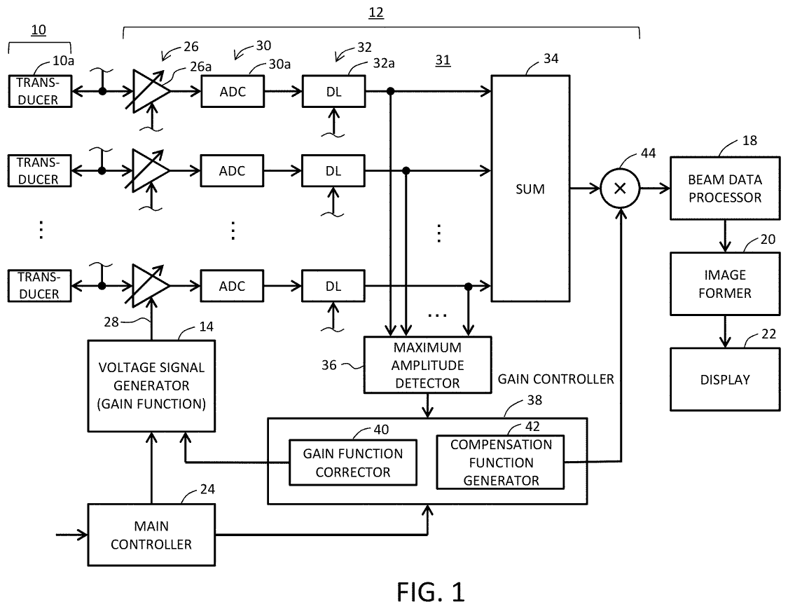

FIG. 1 illustrates an example configuration of an ultrasonic diagnostic apparatus according to an embodiment. The ultrasonic diagnostic apparatus is a medical apparatus used in hospitals, for example, for ultrasonic inspection of a subject.

The ultrasonic diagnostic apparatus includes a transducer array 10 of a plurality of transducers 10a arranged linearly within an ultrasonic probe. The transducer array 10 forms an ultrasound beam and electronically scans the ultrasound beam. The electronic scanning methods include an electronic linear scanning method and an electronic sector scanning method, for example. A two-dimensional transducer array may be disposed within the ultrasonic probe to acquire volume data from within the living body.

The transducer array 10 is connected with a transmitter unit, which is not shown, and is also connected with a receiver unit 12 which is shown. The receiver unit 12 is an electronic circuit that processes a plurality of received signals output from the plurality of transducers to thereby generate beam data. A plurality of beam data items arranged along the electronic scanning direction form reception frame data. Each set of beam data is composed of a plurality of echo data items arranged in the depth direction.

A beam data processor 18 is an electronic circuit that processes each set of beam data, and includes a detection circuit and a logarithmic transformer, for example. An image former 20 generates display frame data based on the reception frame data, and includes a digital scan converter (DSC). The display frame data are tomographic image data. A display 22 displays a tomographic image. The display 22 may display other ultrasound images.

In the embodiment, the receiver unit 12 includes an amplifier array 26, an ADC array 30, a delay unit array 32, an adder 34, and a compensator 44. The receiver unit 12 further includes a voltage signal generator 14.

The amplifier array 26 includes a plurality of amplifiers 26a that are variable analog amplifiers. The voltage signal generator 14 outputs voltage signals 28 in parallel, to the plurality of amplifiers 26a. The voltage signal generator 14 includes a gain function designated or selected by a main controller 24 which will be described below. The gain function increases the gain with an increase in the echo generation depth. The voltage signal 28 corresponds to the gain function. Each amplifier 26a amplifies each received signal in accordance with the gain function.

The ADC array 30 includes a plurality of analog-digital converters (ADCs) 30a. The delay unit array 32 includes a plurality of delay units 32a. Each delay unit 32a applies delay or delay processing to the received signal. Delay time data are supplied to the individual delay units 32a. The adder 34 adds up the plurality of delayed received signals to thereby generate beam data. In the illustrated example configuration, the delay unit array 32 and the adder 34 form a beamformer 31.

The individual received signals may be sampled at high speed to generate digital data and the digital data may be stored in a memory and then retrieved from the memory. In this configuration, the retrieving timing is adjusted to thereby adjust the delay time applied to each set of digital data. A plurality of digital data items may be weighted before or after the delay.

The compensator 44 is a circuit that applies gain compensation to the beam data. Specifically, the compensator 44 amplifies the beam data in accordance with a compensation function which will be described below. The function and operation of the compensator 44 will be described in detail below.

In the example configuration illustrated in FIG. 1, a maximum amplitude detector 36 samples the delayed received signals at each detection timing, and detects the maximum amplitude from among the sampled amplitudes. A plurality of maximum amplitudes arranged in the depth direction can be acquired by repeating detection of the maximum amplitude for each detection timing, and these maximum amplitudes form a maximum amplitude sequence.

A representative value other than the maximum value, such as a mean value, a median value, or a minimum value, may be detected at each detection timing.

While only the received signals corresponding to the receiving aperture may be referenced to detect the maximum amplitude, in the embodiment, all received signals output from the transducers 10a are referenced. This configuration according to the embodiment thus enables acquisition of information from a wide range within the living body.

A gain controller 38 corrects the gain function based on the maximum amplitude sequence, and also generates a compensation function. Specifically, the gain controller 38 includes a gain function corrector 40 and a compensation function generator 42. The gain function corrector 40 corrects the gain function that is preset for the voltage signal generator 14 based on the maximum amplitude sequence. More specifically, whether the gain is excessively large or excessively small is determined based on the maximum amplitude for each depth, and, in response to determination of the gain being excessively large or excessively small, the gain function corrector 40 corrects the gain. The gain function corrector 40 then outputs a signal indicative of correction of the gain function to the voltage signal generator 14.

The compensation function generator 42 generates the compensation function that is complementary to the corrected portion of the gain function and outputs the compensation function to the compensator 44. When the gain in the amplifier array 26 is lowered by the gain function, the gain in the compensator 44 is raised. In contrast, when the gain in the amplifier array 26 is raised by the gain function, the gain in the compensator 44 is lowered. Thus, the gain compensation to the beam data enhances the quality of the beam data.

The main controller 24 controls the operation of components within the ultrasonic diagnostic apparatus. In the embodiment, the main controller 24, prior to the ultrasonic inspection, selects, among a plurality of gain functions, a gain function to be used, or the original gain function, in accordance with various conditions, including the ultrasonic probe and the transmission frequency, for example, and sets the selected gain function for the voltage signal generator 14.

The main controller 24 is composed of a CPU that executes a program, for example. Each of the beamformer 31, the compensator 44, the gain controller 38, and the maximum amplitude detector 36, for example, may be composed of a processor. The processor may be a device such as a field-programmable gate array (FPGA).

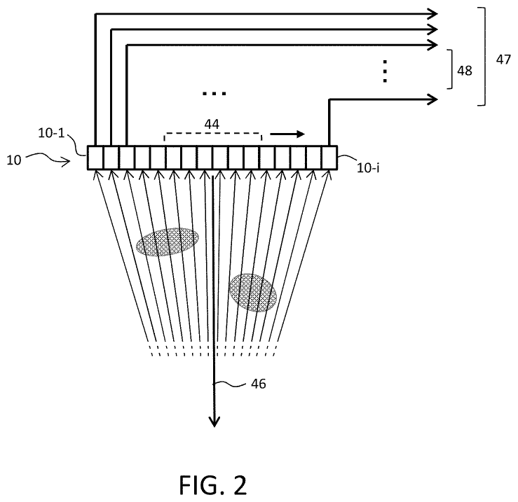

FIG. 2 illustrates the transducer array 10 including transducers 10-1 to 10-i arranged linearly. The transducer array 10 may include a plurality of transducers arranged in an arc shape. Reference numeral 44 denotes a transmitting and receiving aperture.

At the time of transmitting, a transmitting beam 46 is formed using the transmitting aperture. Specifically, a plurality of transducers within the transmitting aperture emit ultrasonic waves into the living body. At the time of receiving, each of the transducers 10-1 to 10-i receives a reflection wave from within the living body. More specifically, reflection waves generated at various points within the living body reach the respective transducers 10-1 to 10-i. The transducers 10-1 to 10-i then output respective received signals.

Reference numeral 47 denotes a received signal sequence including all received signals. In the embodiment, the received signal sequence 47 is referenced for correcting the gain function. Reference numeral 48 denotes a received signal sequence of received signals corresponding to the receiving aperture. The received signal sequence 48 is used for generating the beam data. However, the received signal sequence 47 may be used to generate the beam data. The received signal sequence 48 may further be referenced for correcting the gain. Parallel reception, which will be described below, may be executed at the time of receiving.

While FIG. 2 schematically illustrates the reflection waves directed toward the transducers 10-1 to 10-i, the reflection waves actually reach the transducers 10-1 to 10-i from all orientations. When a strong reflector is present within the living body, the strong reflector affects all or part of the received signal sequence 47.

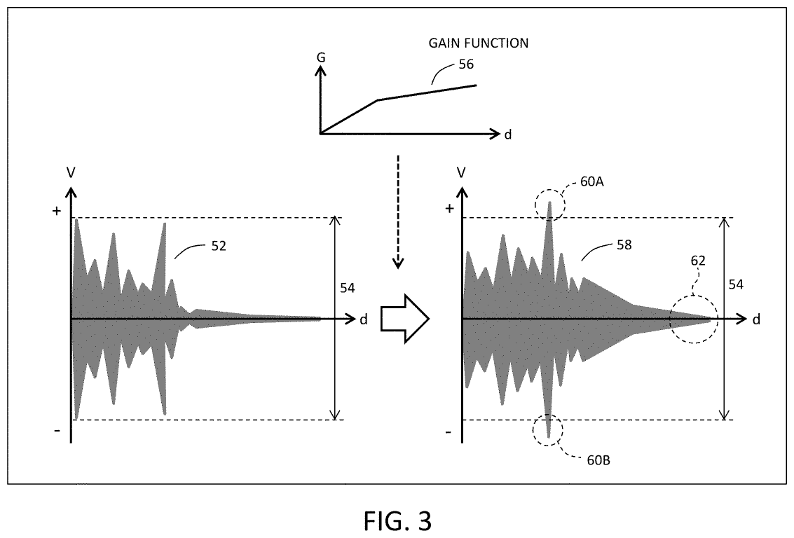

FIG. 3 illustrates the operation of the amplifiers described above. Reference numeral 52 denotes a received signal before amplification. The horizontal axis is a depth axis d indicating the depth, which may also be referred to as a time axis. The vertical axis is an amplitude axis, which is actually a voltage axis V. Reference numeral 54 denotes an input dynamic range of the ADC.

The amplifier amplifies the received signal 52 in accordance with a gain function 56. In the gain function 56, the horizontal axis is a depth axis d and the vertical axis is a gain axis G. The amplified received signal is denoted by reference numeral 58. In the example illustrated in FIG. 3, the received signal 58 includes portions 60A and 60B exceeding the input dynamic range 54 and also includes a portion 62 having a very small amplitude. The portions 60A and 60B cause saturation of the received signal and the portion 62 causes lowering of sensitivity.

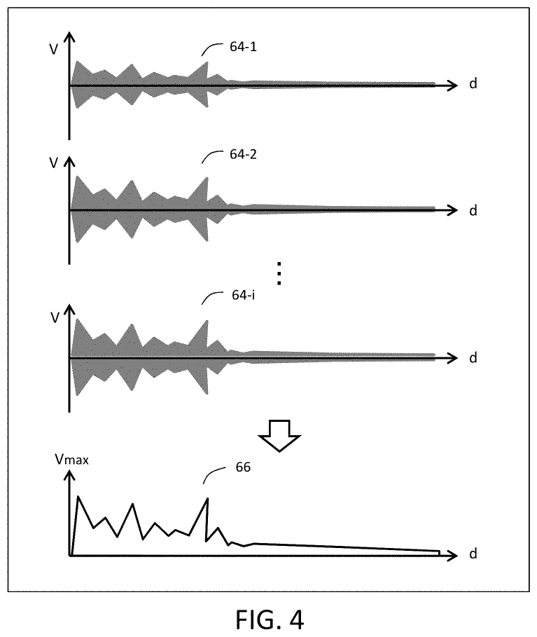

FIG. 4 illustrates a method of detecting the maximum value sequence. Reference numerals 64-1 to 64-i denote amplified and delayed received signals. The maximum amplitude detector 36 samples a plurality of received signals at each detection timing to acquire a plurality of amplitudes, which are, more correctly, a plurality of amplitude absolute values. The maximum amplitude detector then specifies, from among the plurality of amplitudes, the maximum amplitude, which is, more correctly, the maximum amplitude absolute value. A plurality of maximum amplitudes specified at a plurality of detection timings form a maximum amplitude sequence (maximum amplitude profile) 66. The gain function is corrected based on the maximum amplitude sequence 66.

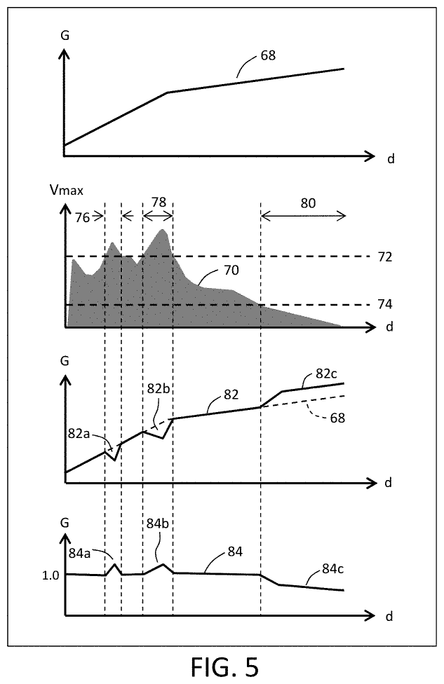

FIG. 5 illustrates a method of generating the corrected gain function and the compensation function. FIG. 5 sequentially shows, from top to bottom, an original gain function 68, a maximum amplitude sequence 70, a corrected gain function 82, and a compensation function 84. The original gain function 68 corresponds to a gain function before correction.

A first threshold value 72 and a second threshold value 74 are set for the maximum amplitude sequence 70. The first threshold value 72 is greater than the second threshold value 74. Each of the maximum amplitudes forming the maximum amplitude sequence 70 is compared with the first threshold value 72 and the second threshold value 74. Reference numeral 76 and reference numeral 78 indicate excessively large portions that exceed the first threshold value 72 in the maximum amplitude sequence 70. Reference numeral 80 indicates an excessively small portion below the second threshold value 74 in the maximum amplitude sequence 70.

In response to occurrence of the excessively large portions 76 and 78, partial downward correction is applied to the original gain function 68, and in response to occurrence of the excessively small portion 80, partial upward correction is applied to the original gain function 68. This results in generation of the corrected gain function 82. The corrected gain function 82 includes recess portions 82a and 82b corresponding to the excessively large portions 76 and 78, respectively, and a raised portion 82c corresponding to the excessively small portion 80. The corrected gain function 82 matching the situation within the living body is thus generated.

The compensation function 84 compensates the gain fluctuation caused by the correction applied to the original gain function 68. Specifically, the compensation function 84 includes protrusion portions 84a and 84b corresponding to the recess portions 82a and 82b, respectively, and a suppressed portion 84c corresponding to the raised portion 82c.

In the embodiment, the corrected gain function and the compensation function generated in the previous transmission and reception are applied to the received signals and the beam data generated in the current transmission and reception. Specifically, the corrected gain function generated in the previous transmission and reception is supplied from the voltage signal generator to each amplifier. Each amplifier amplifies the received signal generated by the current transmission and reception, in accordance with the corrected gain function. Further, the compensation function generated by the previous transmission and reception is applied to the beam data generated by the current transmission and reception. This process is repeated.

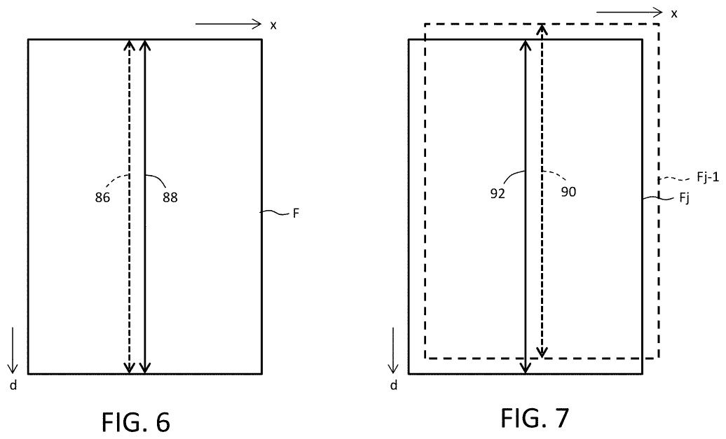

FIG. 6 schematically illustrates the reference relationship according to the embodiment. In FIG. 6, the direction x indicates the electronic scanning direction and the direction d indicates the depth direction. A frame (scan plane) F is formed by a single electronic scanning of the ultrasound beam. Reference numeral 86 denotes the previous transmission and reception and reference numeral 88 denotes the current transmission and reception. The previous transmission and reception 86 and the current transmission and reception 88 are adjacent to each other in terms of time, and the reception signals obtained by the current transmission and reception 88 are controlled by reference to the reception signals obtained in the previous transmission and reception 86.

In the method illustrated in FIG. 6, to execute the initial transmission and reception of the frame F, reference may be made to the last transmission and reception in the frame immediately before the frame F, reference may be made to the transmission and reception at the same location in the frame immediately before as in the frame F, or reference may be made to dummy transmission and reception executed before the initial transmission and reception.

FIG. 7 illustrates another method in which subsequent to a frame Fj−1, a frame Fj is formed. Reference numeral 90 denotes transmission and reception at a certain location in the frame Fj−1, and reference numeral 92 denotes transmission and reception at the same location in the frame Fj. In executing the transmission and reception 92, reference is made to the spatially equivalent transmission and reception 90.

In the method illustrated in FIG. 7, a large storage region need to be secured for storing a plurality of corrected gain functions (and a plurality of compensation functions) corresponding to one frame. Meanwhile, the method illustrated in FIG. 6 needs to secure a storage region for storing only one corrected gain function (and only one compensation function).

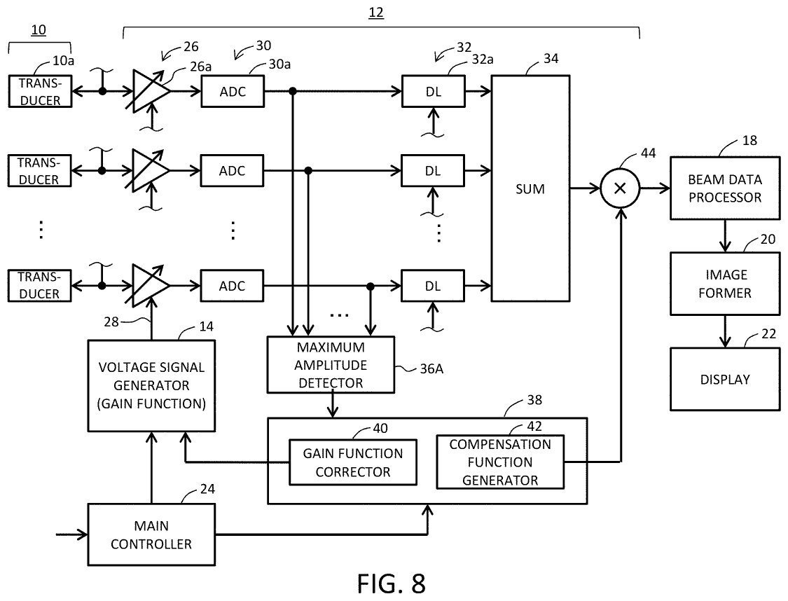

FIG. 8 illustrates a first modification example. In FIG. 8, elements similar to the elements illustrated in FIG. 1 are denoted with similar reference numerals and their descriptions will not be repeated.

In the first modification example, a plurality of received signals after amplification (before delay), output from the ADC array 30, are transmitted to a maximum amplitude detector 36A. The maximum amplitude detector 36A acquires a plurality of amplitudes from the received signal at each detection timing or at each depth, and specifies the maximum amplitude. The plurality of maximum amplitudes acquired at a plurality of detection timings form a maximum amplitude sequence. The first modification example is not affected by a receiving focus in detecting the maximum amplitudes.

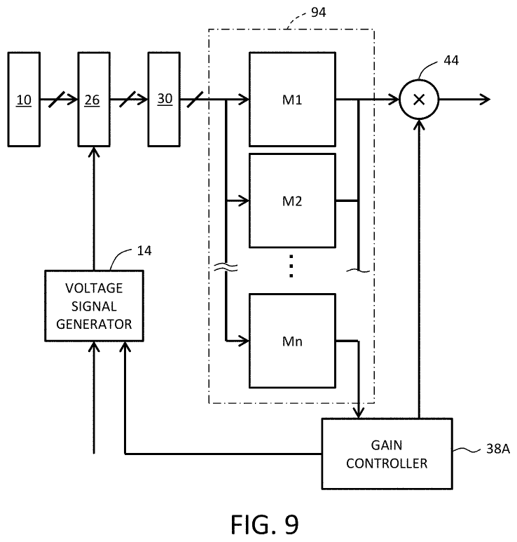

FIG. 9 illustrates a second modification example. In FIG. 9, elements similar to the elements illustrated in FIG. 1 are denoted with similar reference numerals and their descriptions will not be repeated.

A processor 94 includes a plurality of modules M1 to Mn where n represents the number of modules and is 128, for example. Each of the modules M1 to Mn functions as a beamformer. The configuration illustrated in FIG. 9 enables simultaneous formation of a maximum of 128 receiving beams at the time of parallel reception. In practice, only part of the modules M1 to Mn typically operate, whereas the remaining modules are placed in a standby state.

In the second modification example, any one of the modules is used to detect the maximum amplitude. In the example configuration illustrated in FIG. 9, the module Mn functions as a maximum amplitude detector. The module Mn generates a maximum amplitude sequence for each transmission and reception and transmits the generated maximum amplitude sequence to a gain controller 38A.

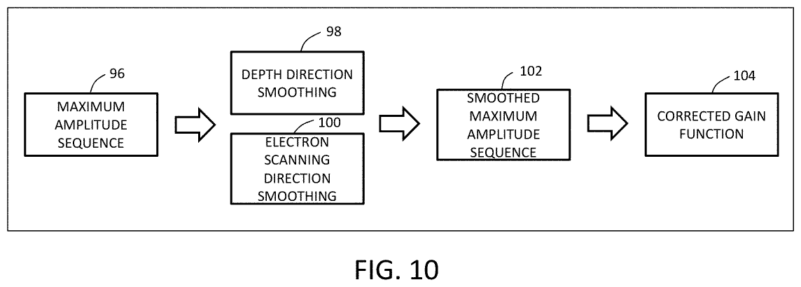

FIG. 10 illustrates a third modification example. In the third modification example, depth direction smoothing 98 and electronic scanning direction smoothing 100 are applied to a maximum amplitude sequence 96 generated by the maximum amplitude detector. Based on a smoothed maximum amplitude sequence 102 thus generated, a corrected gain function 104 is generated. The third modification example can prevent excessive corrections caused by instantaneous amplitude increase or decrease.

The embodiments described above adaptively correct the gain function to thereby enable the gain function to adapt to situations within the living body. The embodiments therefore prevent or reduce saturation of the received signals or lowering of the sensitivity. The configurations of the above embodiments do not generate a new gain function but partially correct the existing gain function. This results in utilization of the existing configurations and lowered possibility of excessive correction.

1. An ultrasonic diagnostic apparatus comprising:
a plurality of amplifiers configured to amplify a plurality of received signals in accordance with a gain function that varies with a depth and to output a plurality of amplified received signals;
a beamformer configured to apply delay to the plurality of amplified received signals and then add up a plurality of delayed received signals, thereby generating beam data;
a detector configured to detect a representative value sequence comprising a plurality of representative values arranged in a depth direction, based on the plurality of amplified received signals or the plurality of delayed received signals; and
a corrector configured to correct an original gain function based on the representative value sequence to thereby generate a corrected gain function, wherein
the corrected gain function is applied to the plurality of amplifiers as the gain function.

2. The ultrasonic diagnostic apparatus according to claim 1, wherein
each of the plurality of representative values is a maximum amplitude detected at each detecting timing, and
the representative value sequence is a maximum amplitude sequence.

3. The ultrasonic diagnostic apparatus according to claim 2, wherein
the corrector is configured to:
determine at least an excessively large gain based on the maximum amplitude sequence; and
in response to determination of the excessively large gain, apply downward correction to the original gain function.

4. The ultrasonic diagnostic apparatus according to claim 3, wherein
the corrector is configured to:
determine an excessively small gain based on the maximum amplitude sequence; and
in response to determination of the excessively small gain, apply upward correction to the original gain function.

5. The ultrasonic diagnostic apparatus according to claim 4, wherein
the corrector is configured to:
determine the excessively large gain based on comparison of respective maximum amplitudes comprising the maximum amplitude sequence with a first threshold value; and
determine the excessively small gain based on comparison of the respective maximum amplitudes comprising the maximum amplitude sequence with a second threshold value that is smaller than the first threshold value.

6. The ultrasonic diagnostic apparatus according to claim 1, wherein
the corrected gain function generated based on a plurality of received signals obtained in previous transmission and reception is applied to a plurality of received signals obtained in current transmission and reception.

7. The ultrasonic diagnostic apparatus according to claim 6, wherein
the previous transmission and reception and the current transmission and reception are continuous in time.

8. The ultrasonic diagnostic apparatus according to claim 1, further comprising:
a generator configured to generate a compensation function based on the representative value sequence; and
a compensator configured to apply gain compensation to the beam data in accordance with the compensation function.

9. The ultrasonic diagnostic apparatus according to claim 8, wherein
the generator generates the compensation function such that an amplitude of the beam data increases in response to application of downward correction to the original gain function and such that an amplitude of the beam data decreases in response to application of upward correction to the original gain function.
